(19) United States
(12) Patent Application Publication
(10) Pub. No.: US 2017/0355944 A1
(43) Pub. Date: Dec. 14, 2017

(54) CULTURE FLASK

(71) Applicant: SANPLATEC CORPORATION LTD., Osaka-shi, Osaka (JP)

(72) Inventors: Satoshi KATOU, Osaka-shi, Osaka (JP); Junichi KUWABARA, Osaka-shi, Osaka (JP); Yuki IWAMOTO, Osaka-shi, Osaka (JP); Hidenori NAKAJIMA, Kyoto-shi, Kyoto (JP); Yoshifumi KOBAYASHI, Kyoto-shi, Kyoto (JP)

(21) Appl. No.: 15/538,560

(22) PCT Filed: Dec. 26, 2014
(86) PCT No.: PCT/JP2014/084620

§ 371 Date: Jun. 21, 2017

(51) Int. Cl. C12M 1/24; C12M 1/00

(52) U.S. Cl. CPC C12M 23/08; C12M 23/22

(57) ABSTRACT

To provide a culture flask A1, a vessel 1 is made up of a bottom wall 11, standing walls rising from the periphery of the bottom wall 11 such as front and rear walls 12, 13 spaced apart in a front-back direction and a pair of side walls 14 laterally spaced and connected to the front and rear walls 12, 13, and a ceiling wall 15 positioned above the bottom wall 11 to cover a region surrounded by the front wall 12, rear wall 13 and paired side walls 14 as viewed in a vertical direction. A cylindrical neck 2 with an open end is attached to the front wall 12. The ceiling wall 15 includes a low region positioned offset in the vertical direction toward the bottom wall 11 from an upper end P1 of the neck 2 attached to the front wall 12.

### TECHNICAL FIELD

The present invention relates to a culture flask used for cell culture, tissue culture and the like.

### BACKGROUND ART

Culture flasks are widely used for cultivation of cells or tissues, and to implement cultivation, a culture medium is put in a flask. A culture flask typically includes a vessel for containing cells or medium, and a neck through which cells or culture medium are putting in and taking out from the vessel (see e.g. Patent Document 1). The vessel has a bottom wall, front and rear walls standing from the periphery of the bottom wall and spaced apart in the front-back direction, a pair of side walls connected to the front and rear walls and spaced apart from each other in the lateral direction, and a ceiling wall facing the bottom wall. The vessel is generally in the form of a hollow box. The neck is attached to the front wall and has a cylindrical shape with an open end. The neck is inclined upward as extending toward the end. A cap is attachable to the neck.

As shown in Patent Document 1, the bottom wall may be in the form of an elongated rectangle, and a bottom inclined wall (inclined surface 36) inclined downward as extending toward the bottom wall is provided between the front wall and the bottom wall. The bottom inclined wall extends downwards from a position adjacent to the lower end of the neck attached to the front wall to reach the bottom wall. To put culture medium into the vessel, the culture medium (liquid) is injected from a pipette that is inserted through the neck to the vicinity of the bottom inclined wall. Since the bottom inclined wall is relatively high near the neck, the supplied medium does not easily flow back toward the neck when the bottom wall is put on the lower side. The paired side walls have portions tapered to the front wall. With this shape of the vessel, every corner above the bottom wall can be reached by a pipette for sucking the culture medium or by a scraper for peeling the cell.

The ceiling wall is positioned higher than the upper end of the neck attached to the front wall. A plurality of culture flasks can be stacked together by letting the ceiling wall of one culture flask support the bottom wall of another culture flask.

When adherent cells are cultured, the cells will proliferate adhering to the culture surface (bottom wall) of a culture flask covered with a certain amount of culture medium. Conventionally, cultured cells are usually transported in a frozen state. In this case, first, the cultured cells need to be put into a special container for cryopreservation and frozen in the container, and then transported. The cultured cells are then thawed and transferred back to a culture container in which the necessary amount of culture medium has been injected. This method may have disadvantages that transferring cells between containers involves the risk of contamination and cell loss and that a lot of time and techniques for freezing/thawing are required to make the cell usable. Recent advances in constant-temperature transportation technology have made it possible to transport cells without freezing the cells while keeping the temperature suitable for culture. By transporting cells while maintaining the culture state, the cells can be immediately used for experiments, research, etc. at the destination. However, in the case of transportation maintaining the culture state, in order to prevent the cells adhered to the bottom wall from peeling off due to possible waving of the culture medium, the inside of the culture flask needs to be entirely filled with the culture medium for suppressing the movement of the culture medium. Thus, such culture-state-maintaining transportation requires a relatively large amount of culture medium to be used.

Depending on the type of cultured cells, the price per unit amount of culture medium is high. In transportation maintaining the culture state, the use of such an expensive culture medium in large amounts leads to an increase in the cost.

### PRIOR ART DOCUMENT

### Patent Document

Patent Document 1: JP Patent No. 2683732

### SUMMARY OF THE INVENTION

### Problems to be Solved by the Invention

The present invention has been proposed under the above-noted circumstances, and an object of the invention is to provide a culture flask suitable for reducing the cost of culture-state-maintaining transportation.

### Means to Solve the Problem

According to the present invention, there is provided a culture flask that may be made up of: a vessel including a bottom wall, a standing wall standing from a periphery of the bottom wall, and a ceiling wall positioned above the bottom wall and covering a region surrounded by the standing wall as viewed in a vertical direction; and a cylindrical neck attached to a predetermined portion of the standing wall and including an open end. The ceiling wall includes a low region that is offset in the vertical direction toward the bottom wall from an upper end of the neck attached to the standing wall.

In a preferred embodiment, the proportion of the low region to the entirety of the ceiling wall is 60% or more in terms of a projected area in the vertical direction.

In a preferred embodiment, the low region includes a first portion disposed at a height position in the vertical direction, where the height position is spaced from a lower end of the standing wall by not greater than one half a dimension of the standing wall in the vertical direction.

In a preferred embodiment, the proportion of the first portion to the entirety of the ceiling wall is 50% or more in terms of a projected area in the vertical direction.

In a preferred embodiment, the standing wall includes a front wall and a rear wall spaced apart from each other in a front-back direction, and a pair of side walls connected to the front wall and the rear wall and spaced apart from each other in a lateral direction. The neck is connected to the front wall.

In a preferred embodiment, the ceiling wall includes: a main ceiling wall portion substantially facing the bottom wall; and a ceiling-side inclined wall portion inclined downward as extending toward the rear wall.

In a preferred embodiment, the ceiling-side inclined wall portion includes a portion flanked by the main ceiling wall portion in the lateral direction.

In a preferred embodiment, the main ceiling wall portion includes an elevated portion positioned relatively higher than other portions and having a dimension in the lateral direction that increases as going toward the rear wall.

In a preferred embodiment, the bottom wall includes a flat main bottom wall portion and an inclined bottom wall portion that is connected to a height position adjacent to a lower end of the neck attached to the front wall and also connected to the main bottom wall portion, where the inclined bottom wall portion is inclined downward as extending from the front wall toward the main bottom wall portion.

In a preferred embodiment, the culture flask may further include a rib projecting downward from the standing wall, and the rib is engageable with an engagement portion formed by an upper end of the standing wall.

In a preferred embodiment, the periphery of the bottom wall is entirely welded to a lower edge of the standing wall.

In a preferred embodiment, the vessel and the neck are formed of a transparent plastic material.

### BRIEF DESCRIPTION OF THE DRAWINGS

FIG. 1 is a perspective view showing an example of a culture flask according to the present invention.

FIG. 2 is a plan view of the culture flask shown in FIG. 1.

FIG. 3 is a sectional view taken along line III-III in FIG. 2.

FIG. 4 is a sectional view taken along line IV-IV in FIG. 2.

FIG. 5 is a perspective view of the culture flask of FIG. 1 as viewed from a bottom side.

FIG. 6 is a perspective view of an upper member before welding as viewed from the bottom side.

FIG. 7 illustrates how the invention works.

FIG. 8 illustrates how the invention works.

FIG. 9 illustrates how the invention works.

FIG. 10 is a perspective view showing another example of a culture flask according to the present invention.

FIG. 11 is a sectional view taken along line XI-XI in FIG. 10.

FIG. 12 is a perspective view showing another example of a culture flask according to the present invention.

FIG. 13 is a sectional view taken along line XIII-XIII in FIG. 12.

### MODE FOR CARRYING OUT THE INVENTION

Preferred embodiments of the present embodiment are described below with reference to the drawings.

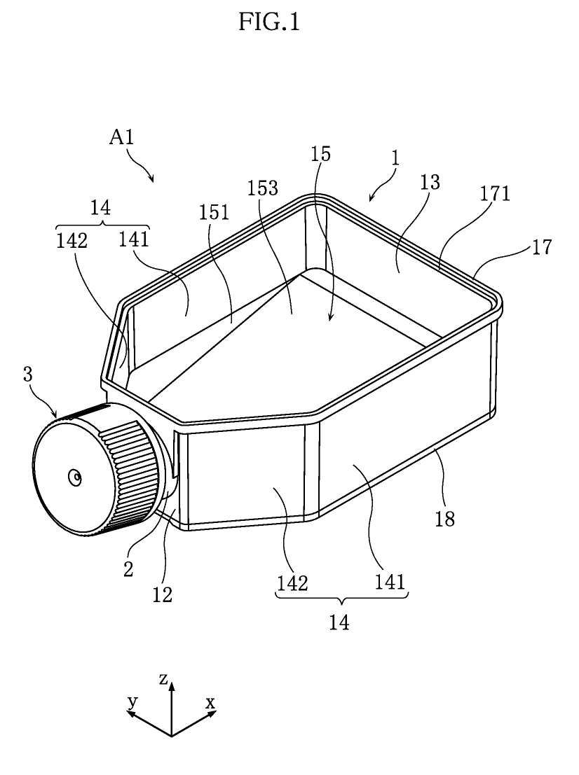

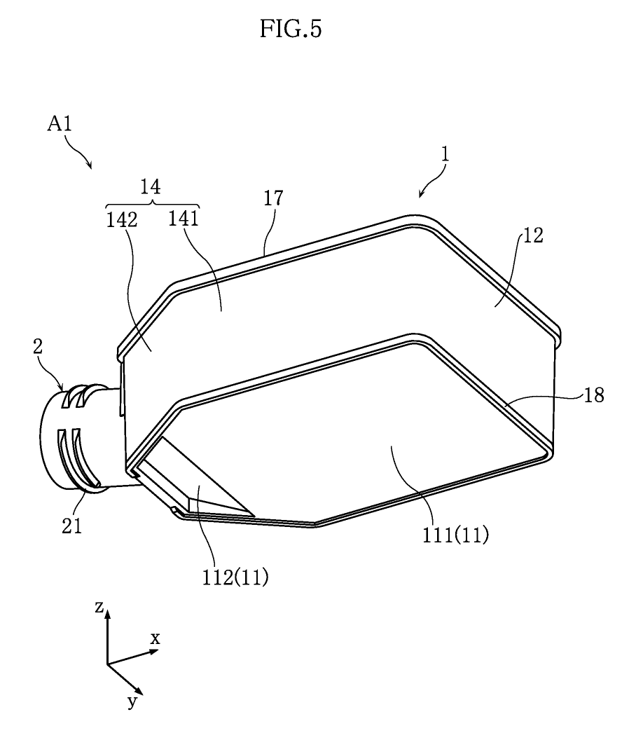

FIGS. 1-5 show an example of culture flask according to the present invention. The culture flask A1 of the present embodiment includes a vessel 1, a neck 2, and a cap 3 for attachment to the neck 2. In Figs . 2-5, illustration of the cap 3 is omitted.

In the present embodiment, the vessel 1 has a bottom wall 11, a front, a rear and a pair of side walls 12, 13 and 14 each standing from the periphery of the bottom wall 11, and a ceiling wall 15. The vessel 1 has an internal space surrounded by these walls.

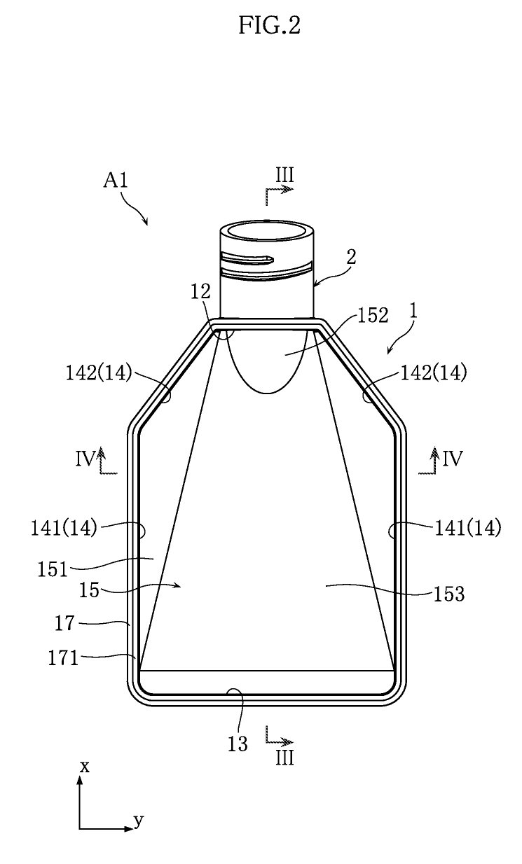

The front wall 12 and the rear wall 13 are spaced apart from each other in the front-back direction (direction x in FIG. 2) and face each other. The front wall 12 is positioned at the front end of the vessel 1, and the rear wall 13 is positioned at the rear end of the vessel 1. The dimension of the front wall 12 in the lateral direction (direction y that is perpendicular to the direction x) is smaller than that of the rear wall 13, and this dimension of the front wall 12 is about 0.4 to 0.5 times that of the rear wall 13, for example. The front wall 12 has an opening at its center, and the neck 2 is connected to the front wall 12 correspondingly in position to the center.

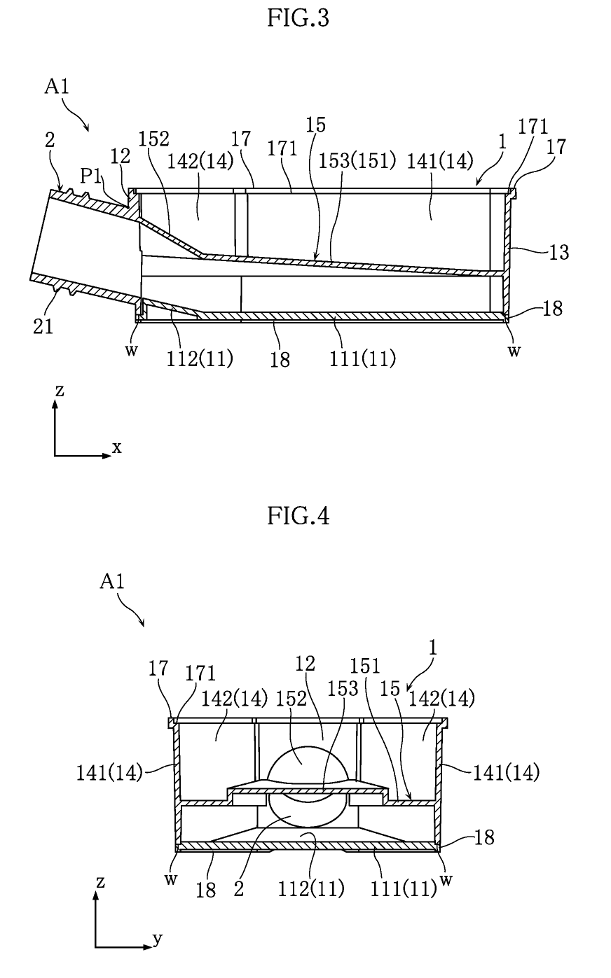

As shown in FIGS. 3-5, the bottom wall 11 has a main bottom wall portion 111 that is flat, and an inclined bottom wall portion 112 that is inclined with respect to the main bottom wall portion 111. The main bottom wall portion 111 extends forward from the rear end of the vessel 1 and is generally in the form of an elongated rectangle. The inclined bottom wall portion 112 is connected to the front wall 12 at a position adjacent to the lower end of the neck 2, while also connected to the main bottom wall portion 111, and is inclined to be displaced downward as extending from the front wall 12 toward the main bottom wall portion 111.

As shown in FIGS. 1-4, the paired side walls 14 are spaced apart from each other in the lateral direction (direction y that is perpendicular to the direction x in FIG. 2) , and each of the side walls has opposite ends connected to the front wall 12 and the rear wall 13, respectively. In the present embodiment, each of the side walls 14 is made up of a main side wall portion 141 connected to the rear wall 13 and a boundary side wall portion 142 connected to the front wall 12. The main side wall portion 141 extends along the periphery of the main bottom wall portion 111. The paired left and right main side wall portions 141 face each other. On the other hand, the paired left and right boundary side wall portions 142 spread apart from each other as extending backward from the front wall 12. The front wall 12, the rear wall 13 and the paired side walls 14 form a standing wall of the present invention.

As shown in FIGS. 1-4, the ceiling wall 15 is positioned above the bottom wall 11 and covers the entire region surrounded by the front wall 12, the rear wall 13 and the paired side walls 14 as viewed in the vertical direction (direction z that is perpendicular to both of the direction x and the direction y). In the present embodiment, the ceiling wall 15 has a main ceiling wall portion 151 generally facing the main bottom wall portion 111 (bottom wall 11), and a ceiling-side inclined wall portion 152. In the present embodiment, as better shown in FIG. 3, compared to the upper end P1 of the connected portion of the front wall 12 to the neck, the ceiling wall 15 (the main ceiling wall portion 151 and the ceiling-side inclined wall portion 152) is provided at lower positions (i.e., closer to the bottom wall 11 in the vertical direction).

In the present embodiment, the main ceiling wall portion 151 includes an elevated portion 153 positioned relatively higher than other parts. The elevated portion 153 is provided over most of the length of the main ceiling wall portion 151 in the front-back direction. The dimension of the elevated portion 153 in the lateral direction varies depending on the position in the front-back direction, and the dimension in the lateral direction increases as going toward the rear wall 13 (deeper side).

Further, in the present embodiment, the remaining part of the main ceiling wall portion 151 other than the elevated portion 153 faces the main bottom wall portion 111 and is flat. On the other hand, the elevated portion 153 is slightly inclined to be displaced downward as extending toward the rear wall 13 (deeper side).

The ceiling-side inclined wall portion 152 is connected to the front wall 12 and the main ceiling wall portion 151 (the elevated portion 153) and inclined to be displaced downward as extending toward the rear wall 13 (deeper side). In the present embodiment, the ceiling-side inclined wall portion 152 is provided by a local part of a cylinder. As shown in FIG. 3, the inclination angle of the ceiling-side inclined wall portion 152 with respect to a horizontal surface is larger than the inclination angle of the elevated portion 153 with respect to a horizontal surface. In the present embodiment, as shown in FIG. 2, the ceiling-side inclined wall portion 152 is flanked by the main ceiling wall portion 151 in the lateral direction (direction y). The main ceiling wall portion 151 extends to the main wall 12 while flanking the ceiling-side inclined wall portion 152. As shown in FIG. 2, the dimension of the ceiling-side inclined wall portion 152 in the lateral direction (direction y) is generally equal to or smaller than the dimension of the neck 2 in the lateral direction . In the present embodiment, the ceiling-side inclined wall portion 152 is generally in the form of a local part of a cylinder, and the dimension of the ceiling-side inclined wall portion 152 in the lateral direction reduces as going toward the rear wall 13 in the front-back direction (direction x).

Regarding the ceiling wall 15, a region thereof that is positioned lower than the upper end P1 of the above-noted connected portion may be referred to as a “low region” of the present invention. For instance, the proportion of the low region to the entirety of the ceiling wall 15 is at least 60%, preferably at least 80%, and more preferably at least 90%, in terms of the projected area in the vertical direction. In the present embodiment, the entirety of the ceiling wall 15 (the main ceiling wall portion 151 and the ceiling-side inclined wall portion 152) is positioned lower than the upper end P1 of the connected portion, and thus the proportion of the low region to the entire ceiling wall 15 is 100%.

The above-noted low region includes an “intendedly low” portion disposed at a predetermined height in the vertical direction. For example, an intendedly low portion may have a height that is equal to or lower than one half the vertical dimension of the front wall 12, the rear wall 13 or the paired side walls 14, with respect to the lower end of the front wall 12, the rear wall 13 or the paired side walls 14. In the present embodiment, the entirety of the main ceiling wall portion 151 corresponds to the intendedly low portion. The intendedly low portion may be referred to as a first portion of the present invention. For instance, the proportion of the intendedly low portion to the entirety of the ceiling wall 15 is at least 50% and preferably at least 80%, in terms of the projected area in the vertical direction. In the present embodiment, the proportion of the intendedly low portion (the main ceiling wall portion 151) to the entirety of the ceiling wall 15 is about 95%.

As understood from FIGS. 2-4, the front wall 12, the rear wall 13 and the paired side walls 14 extend in series along the periphery of the bottom wall 11 as viewed in the vertical direction, forming a closed loop. In the present embodiment, the front wall 12, the rear wall 13 and the paired side walls 14 are substantially equal in height, and the upper edges of the front wall 12, the rear wall 13 and the paired side walls 14 form an engagement portion 17, which is located at a predetermined distance from the main bottom wall portion 111 (the flat portion of the bottom wall 11). The engagement portion 17 is formed with an indented portion 171 on its inner side.

In the present embodiment, a rib 18 is provided so as to project downward from the front wall 12, the rear wall 13 and the paired side walls 14. As understood from FIGS. 3-5, the rib 18 extends along the periphery of the bottom wall 11 as viewed in the vertical direction, forming a closed loop. The rib 18 extends a predetermined distance downward from the main bottom wall portion 111 (flat portion of the bottom wall 11).

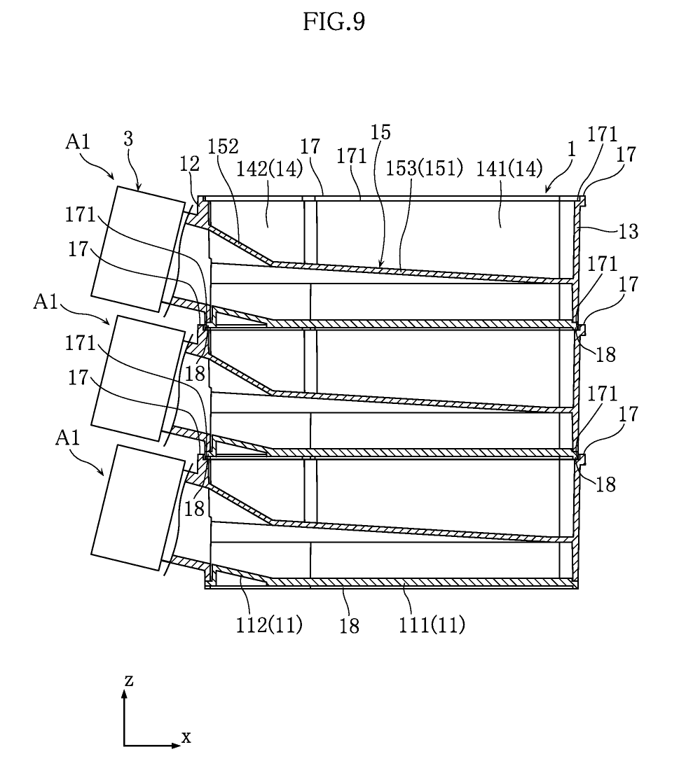

As described in detail below, the rib 18 is configured to fit into the indented portion 171 of the engagement portion 17 in stacking a plurality of culture flasks A1 as shown in FIG. 9. In this way, the engagement portion 17 and the rib 18 are engageable with each other.

The neck 2 is a portion used for putting in and taking out cells or culture medium into and from the vessel 1. The neck 2 is cylindrical and has an opening at its end. The neck 2 is inclined upward as extending toward the end (i.e., away from the front wall 12). The neck 2 is formed with a male thread 21 on its outer surface.

The cap 3 has a cylindrical inner surface formed with a female thread (not shown) to be screwed on the male thread 21 of the neck 2 for closing the end opening of the neck 2. Two types of caps 3, air-permeable and sealable, may be provided and selectively used according to the situation. For example, during cell culture, the air-permeable cap 3 maybe used, though the sealable cap 3 can also be used in a loosened state. At the time of transportation, the sealable cap 3 is used in a tightened state.

Both of the vessel 1 and the neck 2 are formed of a transparent plastic material. Examples of usable transparent plastic material include, without limitation, polystyrene and methyl pentene which are commonly used as medical plastics, as well as other materials, such as cycloolefin polymers and cycloolefin copolymers, which may preferably be transparent.

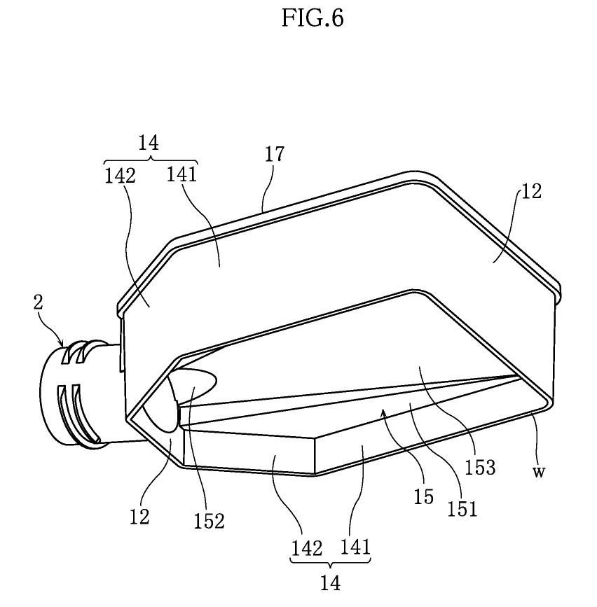

In the present embodiment, the vessel 1 and the neck 2 are obtained as a single article formed by welding two members divided on upper and lower sides. In the present embodiment, the entire periphery of the bottom wall 11 is welded to the respective lower edges of the front wall 12, the rear wall 13 and the paired side walls 14. FIG. 6 is a perspective view of the upper-side members as viewed from the bottom side. As understood from FIGS. 3, 4 and 6, the entire weld surface w is within a single flat plane. Though not illustrated, small projections arranged along the entire weld surface w are formed on the upper-side members or the lower-side member before welding, and these projections are melted during the welding process.

When the culture flask A1 is used for culturing adherent cells, the culture surface (the upper surface of the main bottom wall portion 111) to which cells adhere is subjected to a treatment for providing hydrophilicity by corona discharge or plasma discharge, for example.

The advantages of the culture flask A1 having the above structure are described below.

In the culture flask A1, the ceiling wall 15 of the vessel 1 has a low region located lower than the upper end P1 of the neck 2 connected to the front wall 12. This arrangement substantially reduces the volume of the inner space of the vessel 1, which is defined between the bottom wall 1 and the ceiling wall 15. When adherent cells such as iPS cells are transported while being in a culture state, it is necessary to prevent the peeling of cells adhering to the bottom wall due to waving of the culture medium, and to this end the inside of the culture flask A1 needs to be filled entirely with the culture medium to suppress the movement of the culture medium. According to the present embodiment, the ceiling wall 15 is made relatively low, whereby the amount of expensive culture medium to be used can be reduced for culture-state-maintaining transportation. In addition, the low region of the ceiling wall 15 accounts for a high proportion (100% in the present embodiment) of the size of the entire ceiling wall 15 as viewed in plan, and the intendedly low region of the ceiling wall 15 also accounts for a predetermined high proportion (about 95% in the present embodiment). According to such a structure, the volume of the inner space of the vessel 1 can be greatly reduced as compared with the conventional structure, and a particularly large effect can be expected for culture-state-maintaining transportation with respect to the reduction of the amount of the medium used. Culture-state-maintaining transportation is a very important technology in the field of drug development and regenerative medicine using iPS cells, because it allows iPS cells to be used immediately for experiments, research, etc. at the destination due to the culture state of iPS cells being maintained during transportation. Using culture flasks A1 for such transportation can achieve considerable cost reduction.

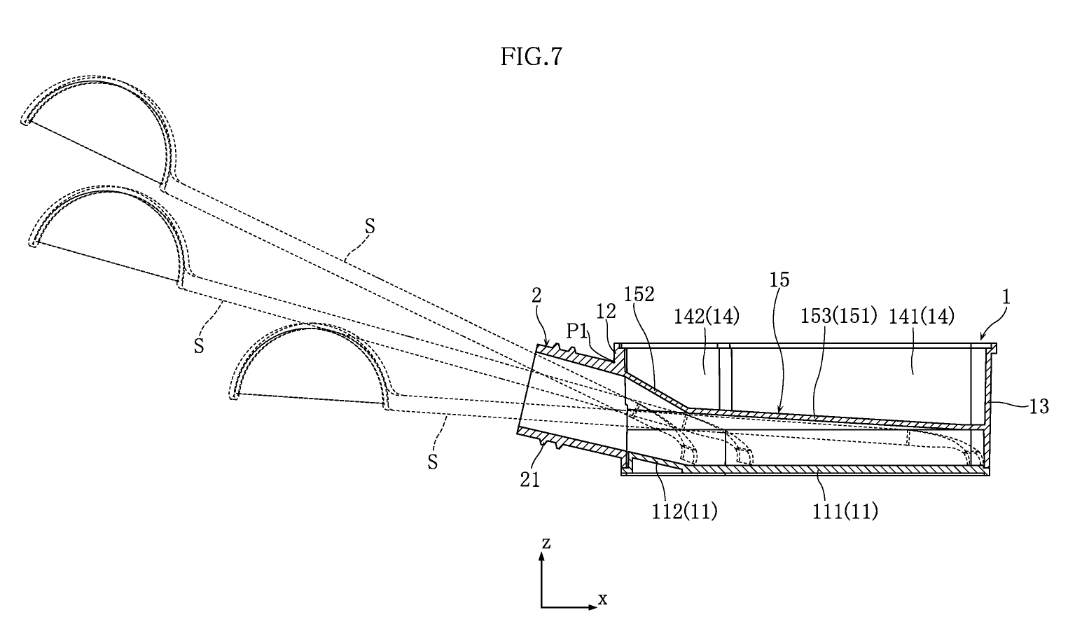

The present embodiment includes the ceiling-side inclined wall portion 152, which is connected to the front wall 12 and the main ceiling wall portion 151 and inclined to be displaced downward as extending toward the rear wall 13. According to this arrangement, in scraping the adhesive cells from the bottom wall 11 (main bottom wall portion 111), it is possible to scrape off the cells over an area covering the deeper side (the rear wall 13 side) as well as the front side (the front wall 12 side) on the main bottom wall portion 111 by using the scraper S (indicated by dashed lines in FIG. 7).

In the present embodiment, the ceiling-side inclined wall portion 152 is flanked by the main ceiling wall portion 151 in the lateral direction (direction x). With such a structure, the proportion of the main ceiling wall portion 151 in the ceiling wall 15 may preferably be as high as possible. It may also be favorable to reduce the volume of the inner space of the container portion 1.

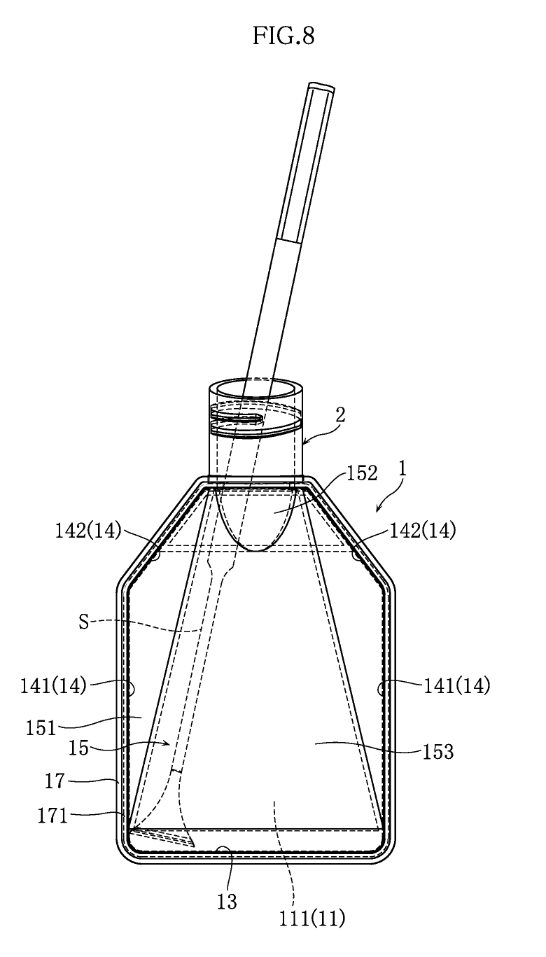

In the present embodiment, the ceiling wall 15 (main ceiling wall portion 151) has an elevated portion 153 which is positioned relatively higher than other portions and of which dimension in the lateral direction (direction y in the figure) increases as going toward the rear wall 13. According to this arrangement, when scraping the adhesive cells from the bottom wall 11 (main bottom wall portion 111), the scraper S can reach corners on a deeper side of the main bottom wall portion 111, as shown in FIGS. 7 and 8. Likewise, a pipette for sucking culture medium can reach corners on a deeper side of the main bottom wall portion 111, so that culture medium can be sucked completely.

In the present embodiment, the elevated portion 153 is inclined downward as extending toward the rear wall 13 (deeper side). Accordingly, the remaining portions of the ceiling wall 15 other than the elevated portion 153 are located at much lower heights. Hence, the volume of the inner space of the vessel 1 can be made advantageously small, which is favorable for reducing the cost for culture-state-maintaining transportation.

The vessel 1 and the neck 2 are provided as a single article formed by welding two upper and lower members. The entire periphery of the bottom wall 11 is welded to the weld surface w or the lower edges of the front wall 12, rear wall 13 and paired side walls 14. According to this structure in which the lower edge of each wall and the periphery of the bottom wall 11 are joined along the weld surface w (single flat plane), it is possible to apply even pressure to the entire weld surface w during welding, which contributes to enabling proper sealing of the vessel 1 by welding.

The rib 18 is provided so as to project downward from the front wall 12, the rear wall 13 and the paired side walls 14. The rib 18 extends a predetermined distance downward from the main bottom wall portion 111 (flat portion of the bottom wall 11). The engagement portion 17, constituted of the upper edges of front wall 12, the rear wall 13 and the paired side walls 14, has a substantially uniform height and is located at a predetermined distance from the main bottom wall portion 111 (the flat portion of the bottomwall 11). The engagement portion 17 and the rib 18 are engageable with each other. This allows a plurality of culture flasks A1 to be stacked as shown in FIG. 9. In the stacked state, the main bottom wall portions 111 of the respective culture flasks A1 are parallel to each other. Thus, it is possible to stack many culture flasks A1 in a stable manner. This contributes to, for example, reduction of the space occupied by the culture flasks A1 in an incubator during culture.

Although the culture flask A1 maintains the ceiling wall 15 relatively low as described above in order to greatly reduce the amount of the culture medium for culture-state-maintaining transportation, operation of a scraper or a pipette for the flask, stacking properties and the like do not differ from those for typical flasks. Thus, the present culture flask can be used without changing the way of handling to which many researchers are accustomed.

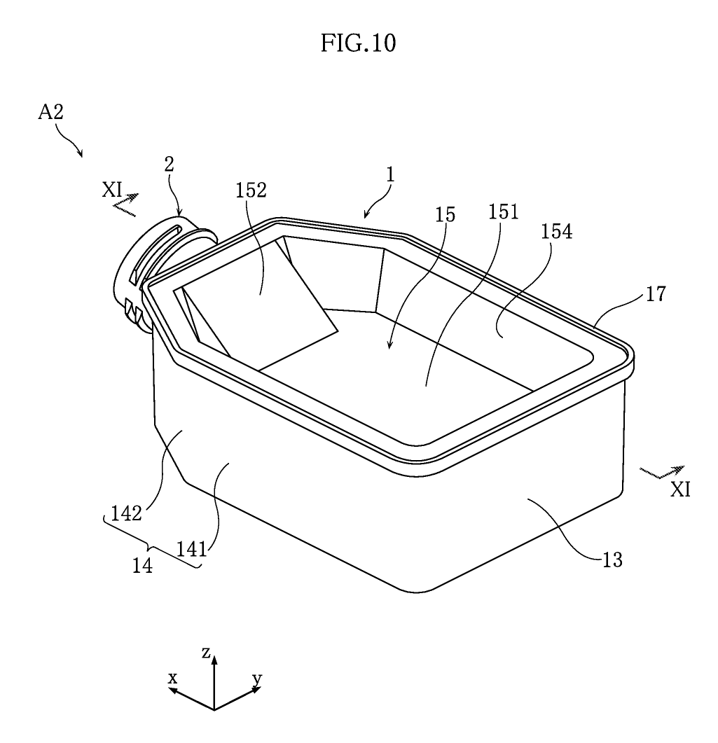

FIGS. 10-13 illustrate another example of culture flask according to the present invention. In these figures, the elements that are identical or similar to those of the foregoing embodiment are designated by the same reference signs as those used for the foregoing embodiment, and the description is omitted appropriately. In FIGS. 10-13, illustration of the cap 3 described in the foregoing embodiment is omitted.

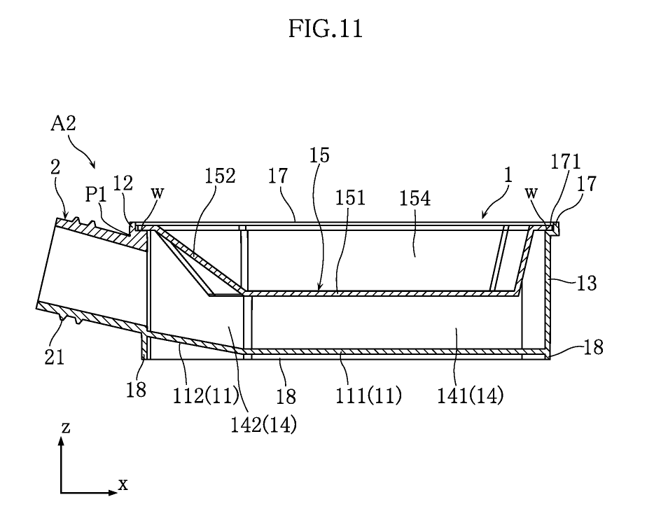

The culture flask A2 shown in FIGS. 10 and 11 has a weld surface w adjacent to the upper edges of the front wall 12, rear wall 13 and paired side walls 14, and hence, has other differences from the culture flask A1.

As understood from FIG. 11, the lower one of the two welded members includes the bottom wall 11, the front wall 12, the rear wall 13, the paired side walls 14 and the neck 2. The upper one of the two welded members includes the ceiling wall 15. The ceiling wall 15 includes a main ceiling wall portion 151, a ceiling-side inclined wall portion 152 and a supporting peripheral portion 154. The main ceiling wall portion 151 is almost entirely flat. The supporting peripheral portion 154 stands from the periphery of the flat main ceiling wall portion 151 and supported on the upper edges of the front wall 12, rear wall 13 and paired side walls 14. As understood from FIG. 11, the entire weld surface w is within a single flat plane. The ceiling-side inclined wall portion 152 is inclined downward as extending toward the rear wall 13 (deeper side), and is flat.

The entirety of the main ceiling wall portion 151, most part of the ceiling-side inclined wall portion 152 and most part of the supporting peripheral portion 154 are positioned lower than the upper end P1 of the neck 2 connected to the front wall 12, and referred to as a low region of the invention. In the present embodiment, the proportion of the low region to the entirety of the ceiling wall 15 is about 92% in terms of the projected area in the vertical direction. Moreover, in the present embodiment, the entirety of the main ceiling wall portion 151 is at a height position corresponding to or lower than one half the dimension of the front wall 12, the rear wall 13 and the paired side walls 14 in the vertical direction from the lower ends of these walls, and constitutes the intendedly low portion in the vertical direction (corresponding to a first portion of the invention). In the present embodiment, the proportion of the intendedly low portion (the main ceiling wall portion 151) to the entirety of the ceiling wall 15 is about 80%.

In the culture flask A2, the ceiling wall 15 of the vessel 1 has a low region (the entirety of the main ceiling wall portion 151, most part of the ceiling-side inclined wall portion 152 and most part of the supporting peripheral portion 154) positioned lower than the upper end P1 of the neck 2 connected to the front wall 12. According to the culture flask A2 in which the ceiling wall 15 is made relatively low, the amount of culture medium used can be reduced for culture-state-maintaining transportation. Thus, by using the culture flask A2, the cost for culture-state-maintaining transportation is reduced.

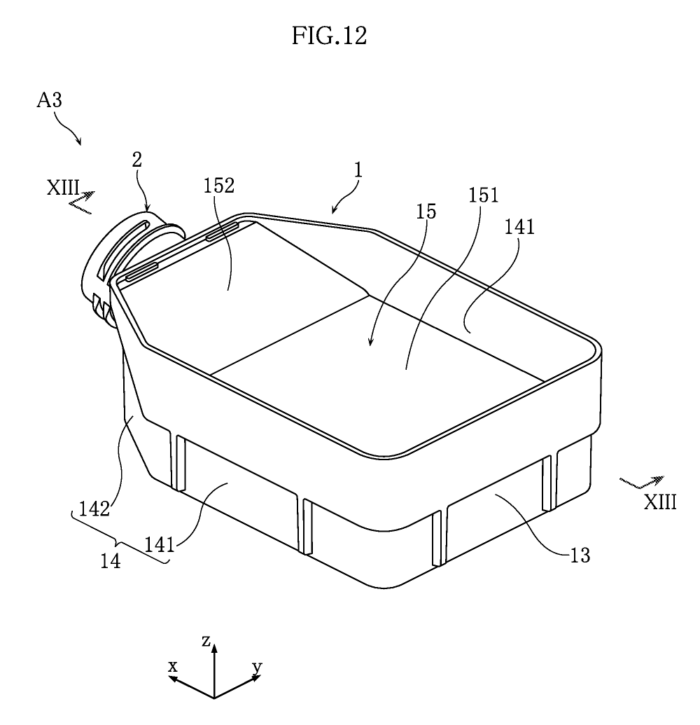

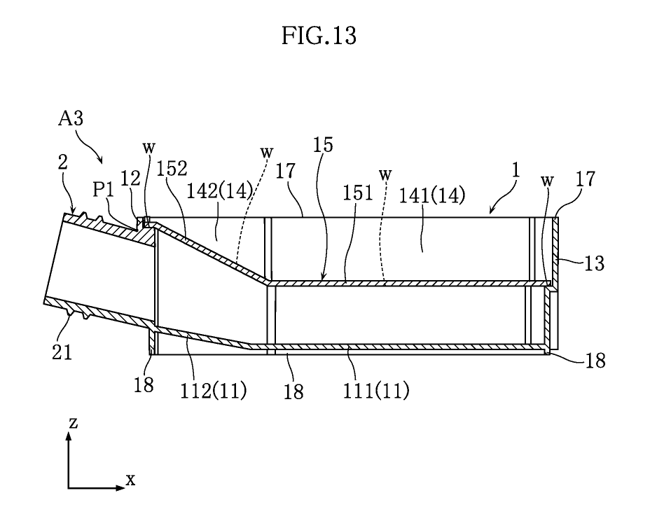

The culture flask A3 shown in FIGS. 12 and 13 has a weld surface w at a position different from that in the culture flask A1, and has other differences from the culture flask A1.

As understood from FIG. 13, the lower one of the two welded members includes the bottom wall 11, the front wall 12, the rear wall 13, the paired side walls 14 and the neck 2. The upper one of the two welded members includes the ceiling wall 15. The ceiling wall 15 includes a main ceiling wall portion 151 and a ceiling-side inclined wall portion 152. The main ceiling wall portion 151 is almost entirely flat.

Each of the front wall 12, the rear wall 13 and paired side walls 14 has a stepped portion in the middle in the height direction, and the portions above the stepped portions are offset outward. The stepped portions of the front wall 12, the rear wall 13 and the paired main side wall portions 141 are horizontally flat. The stepped portions of the paired boundary side wall portions 142 are inclined upward as extending toward the front wall 12 and connected to the stepped portion of the front wall 12. In the culture flask A3, the stepped portions of the front wall 12, the rear wall 13 and the paired side walls 14 constitute a weld surface w. The weld surface w is not entirely within a single flat plane. The ceiling-side inclined wall portion 152 is inclined downward as extending toward the rear wall 13 (deeper side) and is flat.

The entirety of the main ceiling wall portion 151 and most part of the ceiling-side inclined wall portion 152 are positioned lower than the upper end P1 of the neck 2 connected to the front wall 12, and referred to as a low region of the present invention. In the present embodiment, the proportion of the low region to the entirety of the ceiling wall 15 is about 97% in terms of the projected area in the vertical direction. Moreover, of the low region, the entirety of the main ceiling wall portion 151 is at a height position corresponding to or lower than one half the dimension of the front wall 12, the rear wall 13 and the paired side walls 14 in the vertical direction from the lower ends of these walls in the present embodiment, and hence constitutes the intendedly low portion in the vertical direction (corresponding to the first portion of the present invention). In the present embodiment, the proportion of the intendedly low portion (the main ceiling wall portion 151) to the entirety of the ceiling wall 15 is about 85%.

In the culture flask A3, the ceiling wall 15 of the vessel 1 has a low region (the entirety of the main ceiling wall portion 151 and most part of the ceiling-side inclined wall portion 152) positioned lower than the upper end P1 of the neck 2 connected to the front wall 12. According to the culture flask A3 in which the ceiling wall 15 is made relatively low, the amount of culture medium used can be reduced for culture-state-maintaining transportation. Thus, by using the culture flask A3, the cost for culture-state-maintaining transportation is reduced.

Similarly to the culture flask A1, for the culture flasks A2 and A3 again, operation of a scraper or a pipette for the flask, stacking properties and the like do not differ from those for typical flasks, and they can be used without changing the way of handling to which many researchers are accustomed.

Although the embodiments of the present invention have been described above, the scope of the present invention is not limited to the above-described embodiments, and all variations within the scope of the matters described in each claim are included in the scope of the present invention.

In the above-described embodiment, description is made of the paired side walls 14 constituted of paired main side wall portions 141 facing each other and paired boundary side wall portions 142 that spread apart from each other as extending backward. However, the present invention is not limited to such a structure. For example, the paired side walls may be configured such that the entirety of the paired side walls uniformly spread apart from each other as extending backward.

In the above-described embodiments, description is made of a standing wall constituted of the front wall 12, the rear wall 13, and the paired side walls 14, but the configuration of the standing wall is not limited to this. For example, the standing wall may have a generally circular shape as viewed in the vertical direction.

### LIST OF REFERENCE CHARACTERS

A1, A2, A3 Culture flask

P1 Upper end of the connecting portion

w Weld surface

1 Vessel

11 Bottom wall

111 Main bottom wall portion

112 Inclined bottom wall portion

12 Front wall (standing wall)

13 Rear wall (standing wall)

14 Side wall (standing wall)

141 Main side wall portion

142 Boundary side wall portion

15 Ceiling wall

151 Main ceiling wall portion

152 Ceiling-side inclined wall portion

153 Elevated portion

154 Supporting peripheral portion

17 Engagement portion

171 Indented portion

18 Rib

2 Neck

21 Male thread

3 Cap

1. A culture flask comprising:
a vessel including a bottom wall, a standing wall standing from a periphery of the bottom wall, and a ceiling wall positioned above the bottom wall and covering a region surrounded by the standing wall as viewed in a vertical direction; and
a cylindrical neck attached to a predetermined portion of the standing wall and including an open end,
wherein the ceiling wall includes a low region that is offset in the vertical direction toward the bottom wall from an upper end of the neck attached to the standing wall.

2. The culture flask according to claim 1, wherein a proportion of the low region to the entirety of the ceiling wall is 60% or more in terms of a projected area in the vertical direction.

3. The culture flask according to claim 1, wherein the low region includes a first portion disposed at a height position in the vertical direction, the height position being spaced from a lower end of the standing wall by not greater than one half a dimension of the standing wall in the vertical direction.

4. The culture flask according to claim 3, wherein a proportion of the first portion to the entirety of the ceiling wall is 50% or more in terms of a projected area in the vertical direction.

5. The culture flask according to claim 1, wherein the standing wall includes a front wall and a rear wall spaced apart from each other in a front-back direction, and a pair of side walls connected to the front wall and the rear wall and spaced apart from each other in a lateral direction, and
the neck is connected to the front wall.

6. The culture flask according to claim 5, wherein the ceiling wall includes: a main ceiling wall portion substantially facing the bottom wall; and a ceiling-side inclined wall portion inclined downward as extending toward the rear wall.

7. The culture flask according to claim 6, wherein the ceiling-side inclined wall portion includes a portion flanked by the main ceiling wall portion in the lateral direction.

8. The culture flask according to claim 6, wherein the main ceiling wall portion includes an elevated portion positioned relatively higher than other portions and having a dimension in the lateral direction that increases as going toward the rear wall.

9. The culture flask according to claim 5, wherein the bottom wall includes a flat main bottom wall portion and an inclined bottom wall portion that is connected to a height position adjacent to a lower end of the neck attached to the front wall and also connected to the main bottom wall portion, the inclined bottom wall portion being inclined downward as extending from the front wall toward the main bottom wall portion.

10. The culture flask according to claim 1, further comprising a rib projecting downward from the standing wall, and
the rib is engageable with an engagement portion formed by an upper end of the standing wall.

11. The culture flask according to claim 1, wherein the periphery of the bottom wall is entirely welded to a lower edge of the standing wall.

12. The culture flask according to claim 1, wherein the vessel and the neck are formed of a transparent plastic material.
